(19) United States
(12) Patent Application Publication
(10) Pub. No.: US 2015/0119825 A1
(43) Pub. Date: Apr. 30, 2015

(54) OPTIMIZING UNGUAL TREATMENT BY QUANTITATIVE AUTORADIOGRAPHY

(71) Applicant: GALDERMA RESEARCH & DEVELOPMENT, Biot (FR)

(72) Inventors: Hanan Osman-Ponchet, Antibes (FR); Martine Bouclier, Valbonne (FR)

(21) Appl. No.: 14/395,351

(22) PCT Filed: Mar. 15, 2013
(86) PCT No.: PCT/EP2013/055451

§ 371 Date: Oct. 17, 2014

Related U.S. Application Data
(60) Provisional application No. 61/636,240, filed on Apr. 20, 2012.

(51) Int. Cl. A61M 37/00; A61K 31/137

(52) U.S. Cl. CPC A61M 37/00; A61K 31/137; A61M 2037/0007
USPC 604/290; 564/387

(57) ABSTRACT

A method is described for optimizing ungual treatment. The method can include (a) providing a nail having at least two spaced apart holes formed from a surface thereof and extending into the nail, and (b) topically applying a radiolabeled pharmaceutical composition to the nail. At least a portion of the composition is received in the at least two holes. The method also includes (c) sectioning a portion of the nail proximate the at least two holes into a plurality of sections, (d) determining, for each of the plurality of sections, a concentration of radioactivity in the section as a function of position with respect to the at least two holes, and (e) optimizing a spacing between the at least two holes based at least partially on the determinations in step (d). A method of ungual treatment using the optimized spacing is also described.

### BACKGROUND OF THE INVENTION

Embodiments of the present invention relate generally to ungual treatment, and more particularly, to methods for optimization of the treatment.

The nails are frequently the site of fungal infections (onychomycoses), particularly dermatophytic or candidal onychomycoses, or other diseases such as psoriasis or the like. Although the preferred treatment of these pathologies involves local application of pharmaceutical compositions delivered transungually, the rigid structure of the nail makes treatment difficult.

One method of delivering topically applied pharmaceutical compositions into the nail involves the formation of microconduits through the nail such that the agent may diffuse into the nail or nail bed. A device and method for drilling such microconduits are described in U.S. Pat. No. 7,848,799, the entire contents of which are incorporated by reference herein, although other devices and methods exist for this purpose. The microconduits or holes, typically have a depth of about 10% to 100% of the thickness of the nail, which in practice may be about 0.2 to 5 millimeters (mm). The holes also typically have a cylindrical or conical shape, and a diameter between about 400 micrometers (μm) and 1 mm, and more particularly between about 400 μm and 600 μm.

Depending on the size of the infection, often multiple holes need to be formed in the nail to allow the pharmaceutical composition to most effectively treat the affected area. Spacing and orientation of the holes on the nail are critical to successful delivery. It is therefore desired to provide a method for determining an optimal spacing and orientation for holes formed in the nail, which is a function of diffusion of the applied pharmaceutical composition at the nail bed level around each of the holes.

### BRIEF SUMMARY OF THE INVENTION

Briefly stated, an embodiment of the present invention comprises a method for optimizing ungual treatment. The method includes (a) providing a nail having at least two spaced apart holes formed from a surface thereof and extending into the nail, and (b) topically applying a radio labeled pharmaceutical composition to the nail. At least a portion of the radiolabeled pharmaceutical composition is received in the at least two holes. The method further includes (c) sectioning a portion of the nail proximate the at least two holes into a plurality of sections, (d) determining, for each of the plurality of sections, a concentration of radioactivity in the section as a function of position with respect to the at least two holes, and (e) optimizing a spacing between the at least two holes based at least partially on the determinations in step (d).

Another embodiment of the present invention comprises a method of treating an ungual infection in a patient using a topically applied first pharmaceutical composition. The method includes (a) predetermining an optimum spacing between at least two holes according to the method described above using a second pharmaceutical composition. The second pharmaceutical composition is a radiolabeled version of the first pharmaceutical composition. The method further includes (b) forming at least two holes spaced apart by the predetermined optimum spacing in a nail of the patient, and (c) topically applying a therapeutically effective amount of the first pharmaceutical composition to the nail of the patient.

A further embodiment of the present invention comprises a method of treating an ungual infection in a patient comprising the step of topically applying a pharmaceutical composition comprising an antifungal agent on an infected nail provided with at least two holes spaced apart.

### BRIEF DESCRIPTION OF THE DRAWINGS

The foregoing summary, as well as the following detailed description of preferred embodiments of the invention, will be better understood when read in conjunction with the appended drawings. For the purpose of illustration, there are shown in the drawings embodiments which are presently preferred. It should be understood, however, that the invention is not limited to the precise arrangements and instrumentalities shown.

FIGS. 1A-1D are photographs of four nail samples prior to drilling;

FIGS. 2A-2D are photographs of the respective nail samples in FIGS. 1A-1D after drilling and washing;

FIGS. 3A and 3B are autoradiograms of two sections of the nail sample of FIG. 2A with holes spaced 2 mm apart following administration of [¹⁴C]-Terbinafine;

FIG. 4 is a summary graph of total radioactivity in sections of the nail sample of FIG. 2A with holes spaced 2 mm apart following administration of [¹⁴C]-Terbinafine;

FIGS. 5A and 5B are autoradiograms of two sections of the nail sample of FIG. 2A with holes spaced 4 mm apart following administration of [¹⁴C]-Terbinafine;

FIG. 6 is a summary graph of total radioactivity in sections of the nail sample of FIG. 2A with holes spaced 4 mm apart following administration of [¹⁴C]-Terbinafine;

FIGS. 7A and 7B are autoradiograms of two sections of the nail sample of FIG. 2B with holes spaced 2 mm apart following administration of [¹⁴C]-Terbinafine;

FIG. 8 is a summary graph of total radioactivity in sections of the nail sample of FIG. 2B with holes spaced 2 mm apart following administration of [¹⁴C]-Terbinafine;

FIGS. 9A and 9B are autoradiograms of two sections of the nail sample of FIG. 2B with holes spaced 4 mm apart following administration of [¹⁴C]-Terbinafine;

FIG. 10 is a summary graph of total radioactivity in sections of the nail sample of FIG. 2B with holes spaced 4 mm apart following administration of [¹⁴C]-Terbinafine;

FIGS. 11A and 11B are autoradiograms of two sections of the nail sample of FIG. 2C with holes spaced 2 mm apart following administration of [¹⁴C]-Terbinafine;

FIG. 12 is a summary graph of total radioactivity in sections of the nail sample of FIG. 2C with holes spaced 2 mm apart following administration of [¹⁴C]-Terbinafine;

FIGS. 13A and 13B are autoradiograms of two sections of the nail sample of FIG. 2C with holes spaced 4 mm apart following administration of [¹⁴C]-Terbinafine; and

FIG. 14 is a summary graph of total radioactivity in sections of the nail sample of FIG. 2C with holes spaced 4 mm apart following administration of [¹⁴C]-Terbinafine.

### DETAILED DESCRIPTION OF THE INVENTION

Certain terminology is used in the following description for convenience only and is not limiting. The words “right”, “left”, “lower”, and “upper” designate directions in the drawings to which reference is made. The words “inwardly” and “outwardly” refer to directions toward and away from, respectively, the geometric center of the device and designated parts thereof. The terminology includes the above-listed words, derivatives thereof, and words of similar import. Additionally, the words “a” and “an”, as used in the claims and in the corresponding portions of the specification, mean “at least one.”

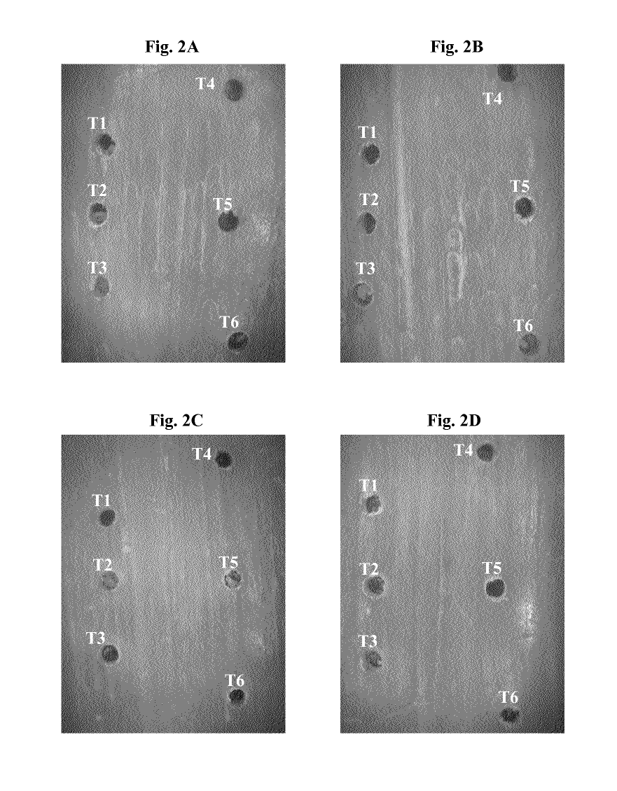

The method described herein calls for the provision of a nail having at least two, and preferably more, spaced apart holes formed from a surface of the nail and extending into the nail. The sample nail is preferably provided by a cadaver, as the nail and nail bed are later sectioned for analysis. It is preferred that multiple sets of holes be formed in the nail, with each set having different spacings between the holes. For example, in FIGS. 2A-2D, the holes T1-T3 of each sample nail are spaced about 2 mm apart, while holes T4-T6 are spaced about 4 mm apart. Other spacings may be utilized in order to make a determination of an optimal spacing for actual patient application. The holes may be formed as described above.

Once the sample nail is prepared, a radio labeled pharmaceutical composition is topically applied to the nail. The radio labeled pharmaceutical composition is preferably the same as the composition expected to be applied to actual patients, but wherein a small percentage of the atoms in the composition, preferably a small percentage of the atoms in the active pharmaceutical ingredient (API) of the composition, are replaced with their radioactive isotopes. Other ingredients, active or inactive, in the composition may also be used for purposes of radiolabeling. Most commonly, carbon-14 (¹⁴C) is used to replace some of the non-radioactive carbon atoms in the pharmaceutical composition. However, other radioactive isotopes can also be used. In the example study described below, Terbinafine, which has proven effective as an antifungal agent in treating onychomycoses, was chosen as the API in the pharmaceutical composition. A small percentage of Terbinafine in the pharmaceutical composition was carbon-labeled prior to application to the sample nails.

It is readily appreciated by those skilled in the art that pharmaceutical compositions comprising other APIs can also be used according to embodiments of the present invention. Pharmaceutical compositions intended to be applied to a perforated nail have been described, see, e.g., WO 2011/073395 A1, the entire contents of which are incorporated by reference herein, although other pharmaceutical compositions can also be used according to embodiments of the present invention.

The topical application of the radiolabeled pharmaceutical composition enables the composition to flow into the holes in the sample nail. From there, the composition diffuses into the nail bed. The method described herein is designed to detect the amount of diffusion that occurs following the topical application. By understanding how the composition behaves upon entering the nail bed, a proper spacing between the holes in the nail of the actual patient can be determined.

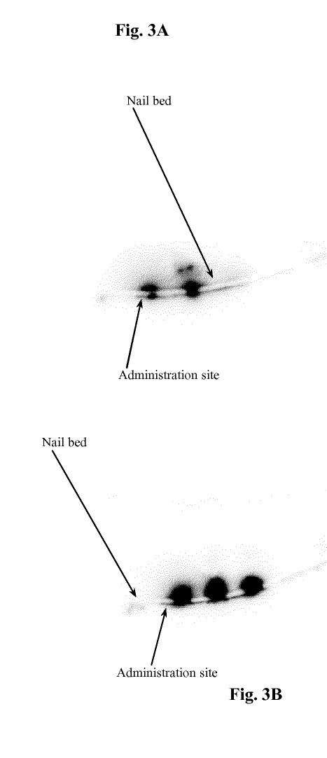

Following application of the radio labeled pharmaceutical composition, the sample nail is sectioned proximate the holes into a plurality of sections using a microtome or like device. The sections are on the order of micrometers thick and may be taken onto adhesive tape. The sections are preferably freeze dried to prevent decomposition during later steps. Preferably, each section encompasses a portion of the nail bed proximate each of the holes in a set. For example, FIGS. 3A and 3B show sections of the nail bed of FIG. 2A which are proximate each of the three holes T1, T2, T3 in the 2 mm spaced set. By encompassing multiple holes in a section, greater efficiency is met in performing the radioactivity detection, and overlap of the diffused composition between holes, to the extent any exists, can be viewed.

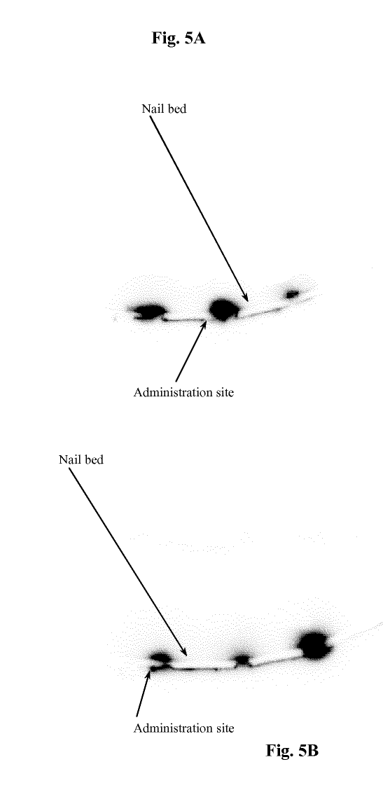

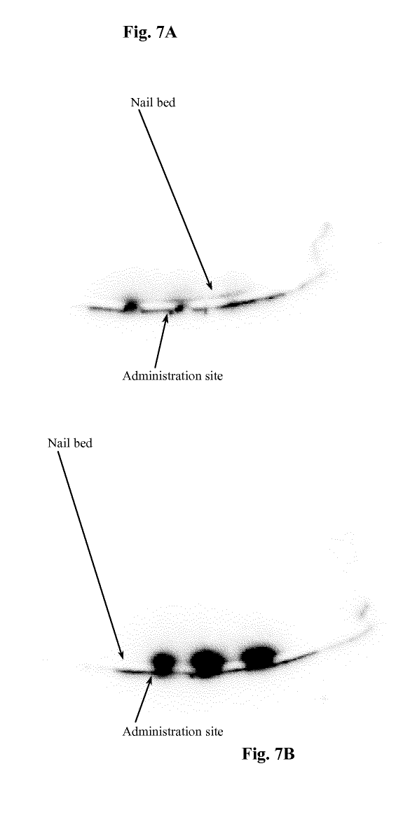

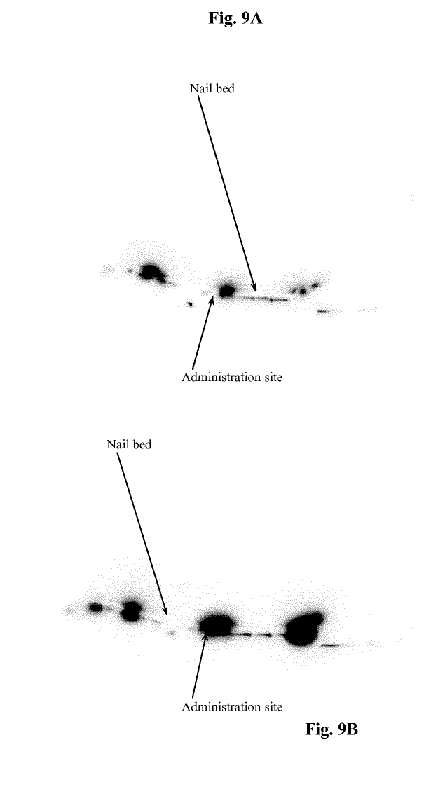

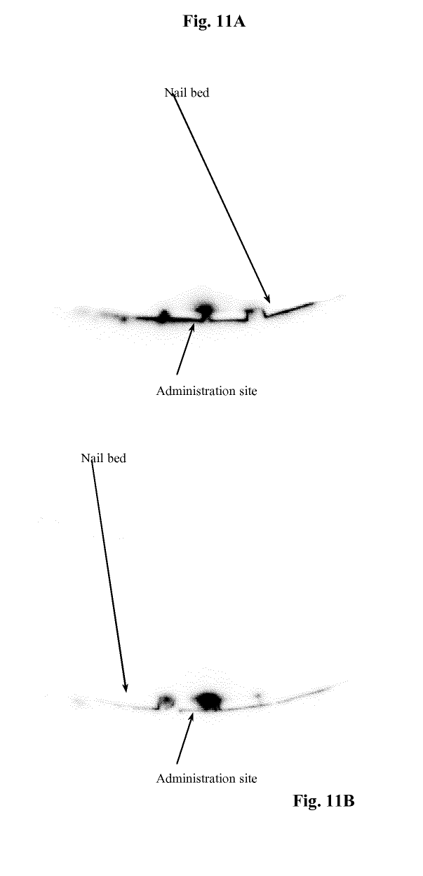

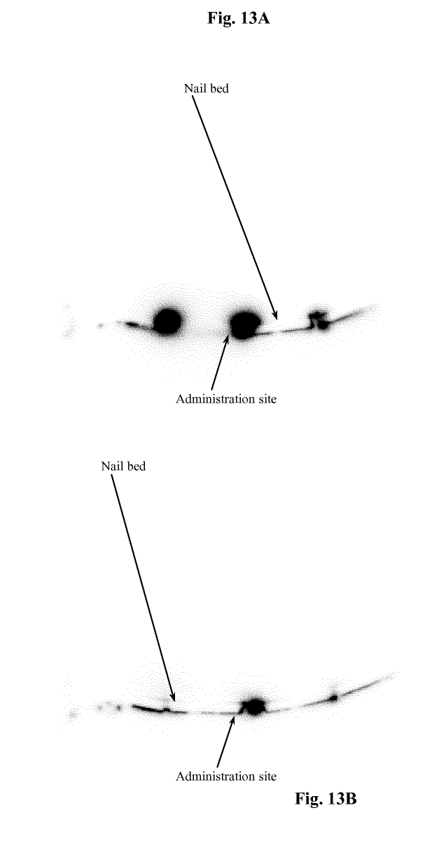

Each of the sections is then analyzed to determine a concentration of radioactivity in the section as a function of position with respect to the holes. It is preferred that quantitative whole body autoradiography (QWBA) is used to make this determination, although other techniques for quantitatively assessing radioactivity levels in tissue may be used as well. For example, the sections may be exposed to a phosphor film or plate for an extended period of time to develop an image from the radiation emitted by the diffused radiolabeled pharmaceutical composition. FIGS. 3A-3B, 5A-5B, 7A-7B, 9A-9B, 11A-11B, and 13A-13B are example resultant autoradiograph images from the samples of FIGS. 2A-2C.

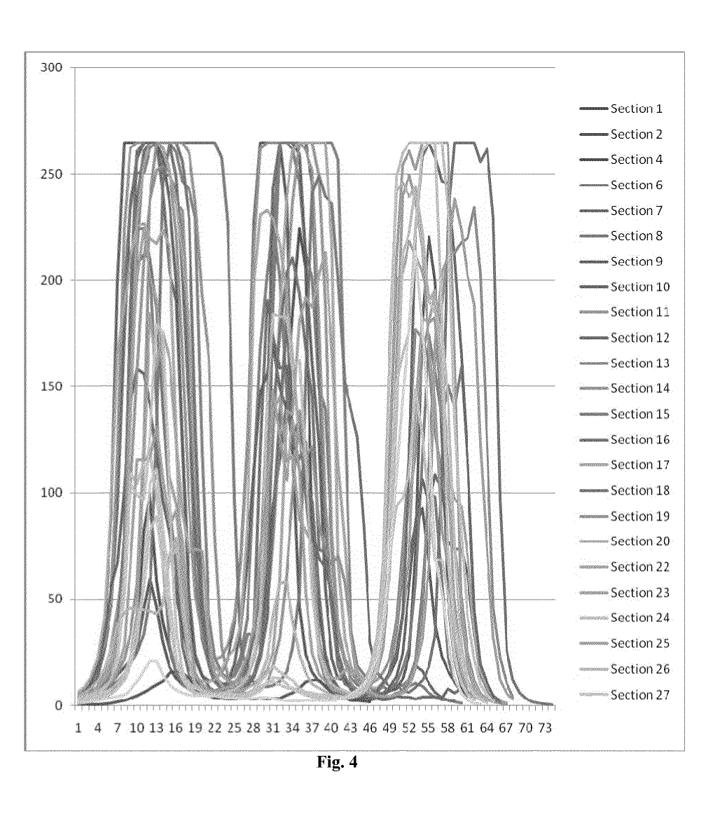

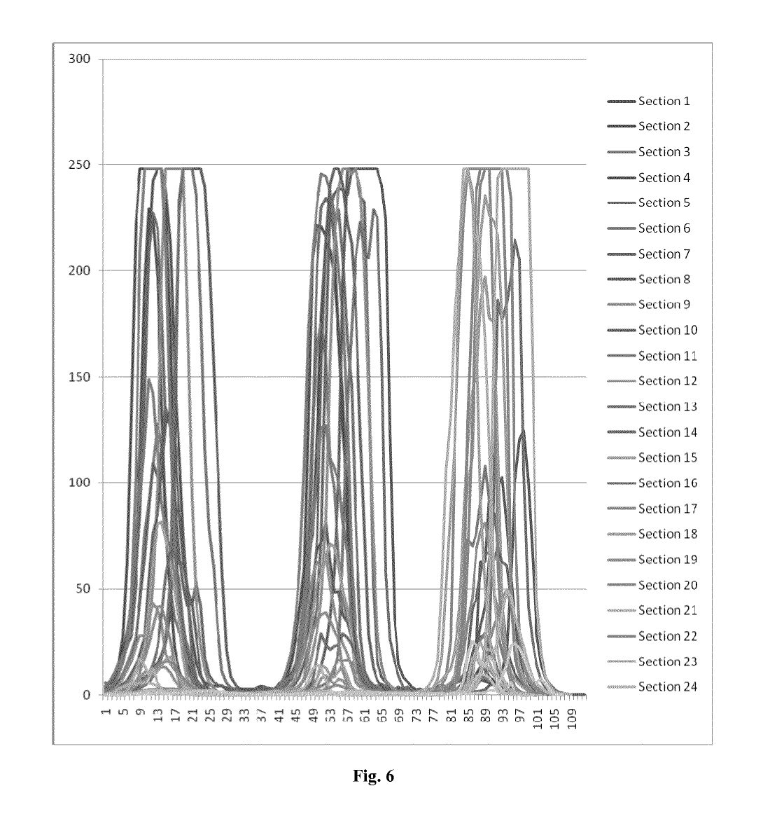

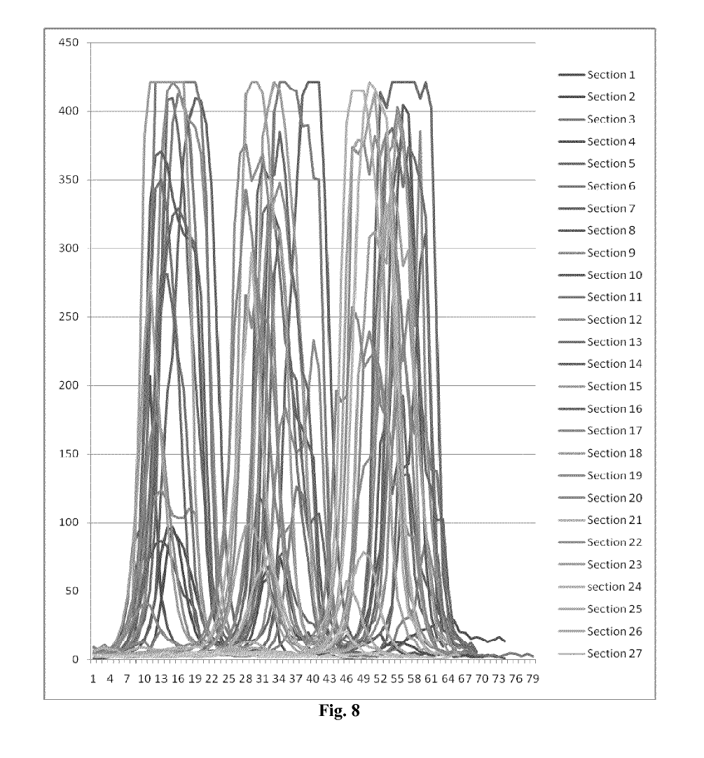

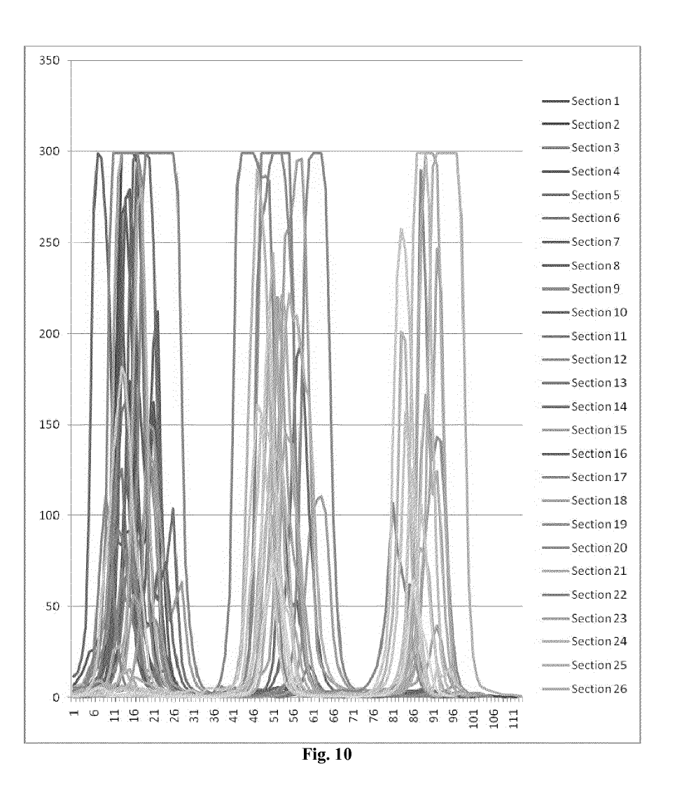

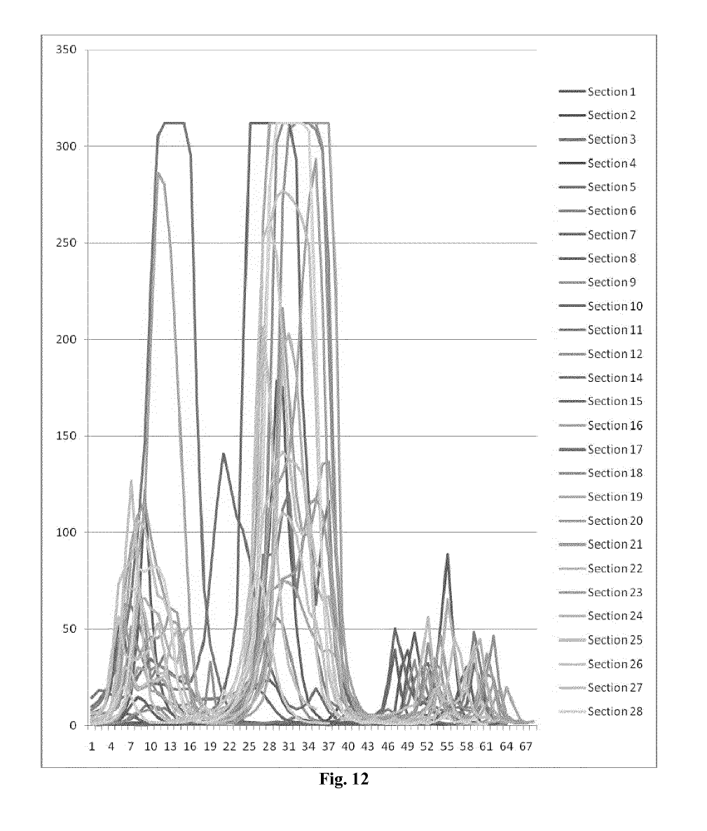

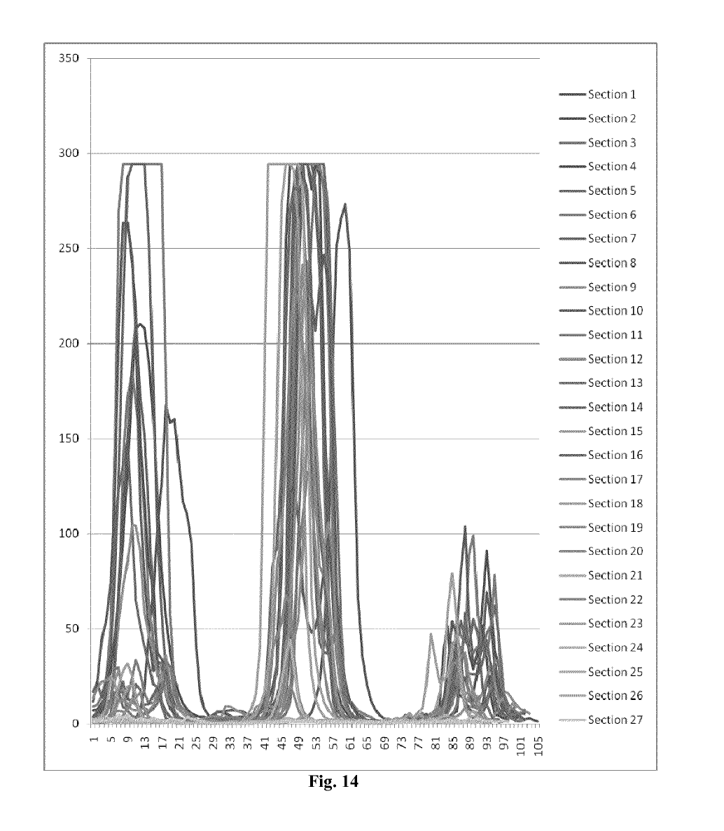

The autoradiographs may be scanned and quantified using image analysis software. The radioactivity levels determined from analysis of the autoradiographs for each section can then be assessed with respect to the position of the holes in the nail. FIGS. 4, 6, 8, 10, 12, and 14 are examples of plots of radioactivity levels for the hole sets in each of the samples shown in FIGS. 2A-2C. It is important when analyzing the radioactivity data to be able to account for and remove background radioactivity levels that are naturally occurring.

With this data, the spacing between the holes can now be optimized. For example, as can be seen from the plots in FIGS. 4, 6, 8, 10, 12, and 14, the diffusion of the Terbinafine is particularly localized to the holes in the nail. Accordingly, the 2 mm spacing is preferred to 4 mm spacing. In combination with other considerations, such as the area of the infection, drill capabilities, amount of composition to be applied, cosmetic considerations, and the like, the spacing of the holes is optimized. Additional studies are conducted to further clarify the precise spacing of holes to effectively treat the ungual infection.

Following this method, the holes can be formed in the nail of a patient according to the predetermined optimized spacing. A therapeutically effective amount of the corresponding non-radiolabeled pharmaceutical composition is then topically applied, the said composition comprising preferably an antifungal agent.

The invention relates also to a method of treating an ungual infection in a patient comprising the step of topically applying a pharmaceutical composition comprising an antifungal agent on an infected nail provided with at least two holes spaced apart. A particularly preferred antifungal agent is terbinafine or a pharmaceutical salt or ester thereof. The at least two holes are preferably provided by using the method as described.

Described below are the results of a study embodying a preferred embodiment of the present invention.

### Example Study

This study was conducted to evaluate the extent of diffusion at the nail bed of [¹⁴C]-Terbinafine topically applied on drilled nails in order to determine the optimal spacing of holes needed to cover the nail bed with the drug using a quantitative whole body autoradiography methodology (QWBA).

### Materials and Methods

Four frozen human cadaver fingertips with nails were used. Each fingernail contained two series of three holes, one set at 2 mm apart and the other set at 4 mm apart.

The samples were placed in a Transwell plate containing 3 milliLiters (mL) of culture medium. Three of the nails received an application of the radiolabeled test item during one hour; one received an application of a non-radiolabeled test item. A finite dose (5 microLiters (μL)) of formulation was applied on the nail surface. Incubation was conducted for 1 hour at 37° C., 5% CO₂ and saturated hygrometry. At the end of the incubation, excess of formulation was removed from the nail surface.

Each sample was frozen onto a support using adapted QWBA procedures. Sections were then taken through each fingernail onto tape and were analyzed using QWBA methods to determine the diffusion of radioactivity at the nail bed level from each site of administration. A 3-dimensional graph of the concentration of radioactivity was then created to visualize the diffusion through the nail matrix.

### Results and Conclusions

A marked radioactive signal was observed on the surface of nail plates. Furthermore, the data showed that there was a diffusion of the radiolabeled drug at the nail bed level around the holes and the extent of diffusion around each holes was measured. In addition, there was little diffusion of radioactivity between the 4 mm spaced holes compared to the 2 mm spaced holes. Indeed, in the 4 mm spaced holes the concentrations of radioactivity decreased to background radiation levels between the holes.

Taken together, the data indicate that the 2 mm spacing hole are more suitable to cover the nail bed with the drug. In this study, QWBA was effective to investigate the penetration through the nail of a radio labeled drug topically applied. Furthermore, this appears to be the first application of QWBA for such type of investigation and QWBA proved its versatility in the investigation of distribution in unusual samples for a “non-standard” purpose.

### Description of the Study

In vitro diffusion of [¹⁴C]-Terbinafine on nail beds was studied on drilled human hand nails. Four samples were used through the study and were drilled as follows:
On each sample: 3 holes of 0.6 mm diameter, with 2 mm distance between each hole and 3 holes of 0.6 mm diameter, with 4 mm distance between each hole
3 samples were treated with 10% [¹⁴C]-Terbinafine in amphoteric solution One sample was treated with placebo
Treatment duration: 1 hour
5 μL of test item were applied on each nail
Incubation at 37° C., 5% CO2 and saturated hygrometry during 1 hour
At the end of the incubation period, the excess of test item was removed and the samples were stored at −80° C. before processing for autoradiography analysis.

### Storage of Biological Samples

Before the treatment, the nail samples were stored at approximately −80° C.

### Test Item

[¹⁴C]-Terbinafine Terbinafine hydrochloride was used having a specific activity determined by mass spectrometry of about 59 millicuries per millimole (mCi/mmol) (about 2.18 gigabecquerels (GBq)/mmol)). The radiochemical purity was approximately 99.8% with a molecular weight of 329.8. The material was stored at approximately −20° C.

### Preparation of Radiolabeled Formulation

A formulation containing [¹⁴C]-Terbinafine used through the study was prepared by adding 10 mL of the 10% compound in amphoteric solution (such as disclosed in WO2011/073395) to 1 mCi of [¹⁴C]-Terbinafine representing 5.78 milligrams (mg). In these conditions, Terbinafine represents 0.58% of the total Terbinafine and was considered negligible.

### Control of the Radioactive Concentration

Before use, the radioactivity of the formulation was measured in triplicate by liquid scintillation counting as follows: 100 μL of the formulation was diluted in 10 mL methanol and 5 mL water. 100 μL of this diluted solution was directly counted by liquid scintillation counting in 10 mL of PICO FLUOR available from Perkin Elmer. The radioactive concentration measured in the formulation was 1 microcurie (μCi)/10 μL.

### Control of the Radioactive Purity

The radioactive purity of the formulation was checked before nail treatment by high-performance liquid chromatography (HPLC) analysis with radioactive detection. The radioactive purity of the formulation was equal to about 92.7%.

### Test System

Four human fingernails obtained from human cadaver hands were used throughout the study. Identification of the nail samples is presented in the table below:

| Sample | | |
|---|---|---|
| identification | Study identification | Treatment |
| 119_OL2 | Sample A | [¹⁴C]-Terbinafine |
| 119_OL4 | Sample B | [¹⁴C]-Terbinafine |
| 123_OL3 | Sample C | [¹⁴C]-Terbinafine |
| 123_OL4 | Sample D (control) | Terbinafine |

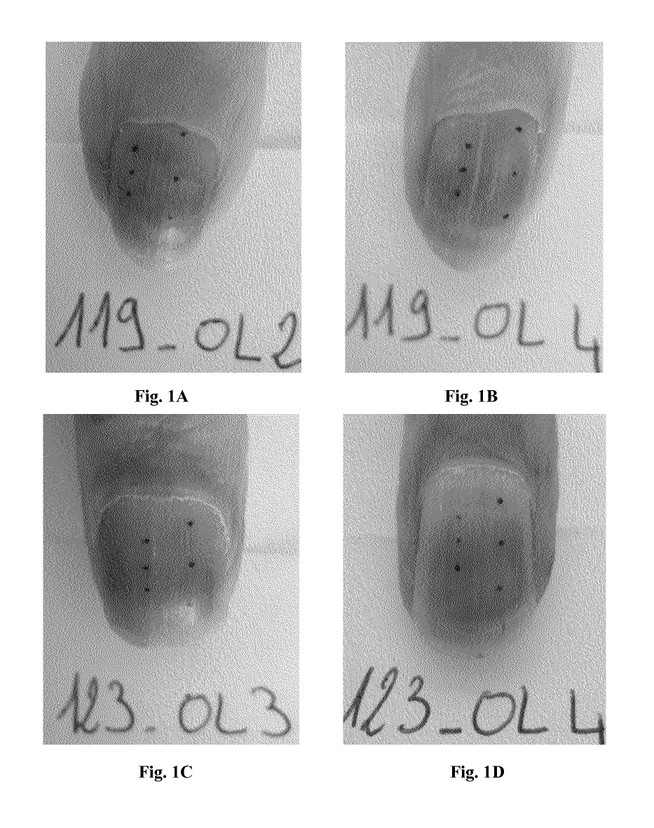

Each fingernail contained 2 sets of three holes (0.6 mm diameter), one set at 2 mm apart and the other set at 4 mm apart as illustrated in FIGS. 1A-1D and 2A-2D.

### Treatment Conditions

After thawing at room temperature, the nail samples were washed once in sodium hypochlorite solution, then three times in HEPES-buffered Hank's balanced salt solution (HHBSS) containing 2% penicilline-streptomycine (v/v). The HHBSS was prepared by adding 1 mM of the HEPES buffer to the HBSS to achieve a final concentration of 0.025 M (25 mL 1 M HEPES to 1 L HBSS).

Each nail sample was then placed on a cell culture insert. Each insert was introduced into a receiver chamber of a 6-well culture plate. The receiver chamber was filled with 3 mL HHBSS containing antibiotics (2% penicilline-streptomycine).

Three of the nails received an application of the radiolabeled test item, whilst the other nail received an application of the unlabelled test item as follows:
Samples A, B and C (Treated samples): 5 μL of the formulation containing 10% [¹⁴C]-Terbinafine were applied on the surface of each of the nail samples.
Sample D (Control sample): 5 μL of the formulation containing 10% Terbinafine were applied on the surface of the nail sample.

The culture plate containing the nail samples was placed in a culture incubator kept at 37° C., 5% CO₂ and saturated hygrometry. The duration of the nail treatment was 1 hour.

At the end of the treatment period, the excess of the formulation remaining on the nail was wiped off using a dried cotton swab and five successive cotton swabs wetted with absolute ethanol. These cotton swabs were discarded. The nail samples were then stored at −80° C., and shipped frozen for analysis by autoradiography.

In order to check the specificity of the incubation system (no radioactive contamination due to leakage of applied formulation through the insert), the radioactivity level was measured in the culture medium at the end of the treatment period. Each receiver chamber medium was controlled in triplicate by liquid scintillation counting as follows: 100 μL of culture medium was directly counted by liquid scintillation counting using 10 mL of HIONIC FLUOR available from Perkin Elmer. The results presented in the table below show a very low radioactivity level in culture medium of sample A and sample C. The radioactivity level only represented 1.6% of the applied dose in sample A and 0.1% of the applied dose in sample C. These radioactivity levels were considered negligible and could not compromise the results of the study.

| | | | Applied dose |
|---|---|---|---|
| Samples | dpm/100 μL | dpm/3 mL | (%) |
| Sample A | 494 | | |
| Sample A | 621 | | |
| Sample A | 612 | | |
| Mean sample A | 575.7 | 17270 | 1.6 |
| Sample B | 48 | | |
| Sample B | 49 | | |
| Sample B | 42 | | |
| Mean sample B | 46.3 | BLQ | 0 |
| Sample C | 49 | | |
| Sample C | 52 | | |
| Sample C | 63 | | |
| Mean sample C | 54.7 | 1640 | 0.1 |
| Sample D | 20 | | |
| Sample D | 14 | | |
| Sample D | 16 | | |
| Mean sample D | 16.7 | BLQ | 0 |

Blank sample = 18 dpm = Background
LOQ = 3 × Background: 54 dpm
Applied dose: 1106175 dpm

### Preparation of Fingernails for Autoradiography

The samples (four human fingernails) were received deeply frozen on solid CO₂ and immediately transferred to storage in a freezer set to maintain a temperature of −80° C. A supporting block was prepared to support each sample during sectioning. Specifically, a mould was filled with 2% carboxymethylcellulose solution and frozen in a mixture of dry ice and hexane. The frozen finger was fixed in a horizontal position on a cork disc using a cryo-matrix and the disc was then fixed to the carboxymethylcellulose block with a further cryo-matrix. The finger was embedded so that the nail was facing to the side and to ensure that each series of holes was as horizontal as possible (dependant on the alignment of the drilled holes). A further cryomatrix was added around each finger to provide additional support during sectioning. The frozen blocks were stored in a freezer set to maintain a temperature of −20° C. prior to and following sectioning.

### Cryo Section

Each nail was sectioned through until immediately adjacent to the uppermost hole in the series. Horizontal sections (approximately 30 μm thick) were then taken through each fingernail onto adhesive tape. Up to thirty sections were taken from each series of holes to allow investigation of the diffusion of radioactivity from each site of administration. Sections were taken using a whole body cryomicrotome (preferably a LEICA CM3600 available from Leica Instruments GmbH). Sections were freeze dried prior to exposure on storage phosphor screens.

### QWBA or Autoradiography

For analysis, the nail sections were placed into close contact with phosphor screens and left for a period of 7 days. On each phosphor screen, a set of external standards was also exposed. These standards were prepared from blood spiked with a serial dilution of a ¹⁴C-labelled reference solution, which was dispensed into holes drilled into a block of carboxymethylcellulose, frozen, and then sectioned in the same way as the nail samples.

The exposed phosphor screens were scanned using a FUJIFILM FLA-5000 Image Analyzer and IMAGEREADER FLA-5000 software available from Fuji Photo Film Co. Ltd. of Japan. After the phosphor screen was scanned, an image of the radioactivity in the sample was stored digitally. The image was then quantified using AIDA image analysis software (available from raytest Isotopenmeβgeräte GmbH of Strubenhardt, Germany) and the levels of radioactivity in each level determined with reference to the appropriate internal and external calibration standards.

For quantitative analysis, six background areas were defined on each storage phosphor screen image. The software automatically calculated the mean background and subsequently subtracted this from all standards and tissues analyzed. A regression coefficient was derived by comparing the response of each standard with the nominal dpm/g over the range of radioactive concentration used and forcing the response curve through the origin. The concentrations of the standards used were in the range of about 0.0071 to 37.1206 nmol equiv/g. The response curve is linear over these concentrations and is assumed to be linear to the limit of reliable determination.

### Data Manipulation

The concentration data obtained from autoradiographic analysis was exported to a spreadsheet. A graphic representation of the distribution in each section was obtained and these were overlaid to provide a summary of the distribution. This summary is presented in both two and three dimensional formats to enable visualization of the distribution.

### Sample Storage

Samples were stored frozen prior to and following analysis in a freezer set to maintain a temperature of −20° C.

### Results

Representative autoradiograms and graphic summarizations of the diffusion of total radioactivity in Nails A to C are presented in FIGS. 3A-14. The vertical (y) axis is concentration (nmol equiv/g) and the horizontal (x) axis represents a distance along the nail. Each “section” is the 30 μm thick slice taken for analysis.

### Nail A

The data shows that both sets of holes in Nail A received an adequate application of radioactivity. There was little diffusion of radioactivity between the holes. This is particularly evident in the 4 mm spacing where concentrations of radioactivity decrease to background levels between the holes.

### Nail B

The data for Nail B is slightly less clear (probably due to the difficulty experienced in precisely aligning the analysis of each section). The data shows that both sets of holes in Nail B received an adequate application of radioactivity. As with Nail A, there was little diffusion of radioactivity between the holes and again this is particularly evident in the 4 mm spacing where concentrations of radioactivity decrease to background levels between the holes.

### Nail C

The data for Nail C indicates that neither set of holes received a full application of radioactivity. The first and last holes in each series (both 2 mm and 4 mm spacing) seem to have less exposure to radioactivity than the middle hole. The last hole in particular has very little radioactivity. The distribution results are therefore more problematic. However, it appears again that there was little diffusion of radioactivity between the holes. Again this is particularly evident in the 4 mm spacing where concentrations of radioactivity decrease to background levels between the holes.

### Nail D

The images obtained from Nail D showed only background concentrations of radioactivity and were therefore not analyzed.

### CONCLUSION

A marked radioactive signal was observed on the surface of nail plates. Furthermore, the data showed that there was a diffusion of the radiolabeled drug at the nail bed level around the holes and the extent of diffusion around each holes was measured. In addition, there was little diffusion of radioactivity between the 4 mm spacing holes compared to the 2 mm spacing holes. Indeed, in the 4 mm spacing holes the concentrations of radioactivity decrease to background levels between the holes.

Taken together, the data indicate that the 2 mm spacing hole are more suitable to cover the nail bed with the drug. In this study, QWBA was effective to investigate the penetration through the nail of radiolabeled drug topically applied. Furthermore, this appears to be the first application of QWBA for such type of investigation and QWBA proved its versatility in the investigation of distribution in unusual samples for a “non-standard” purpose.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method for optimizing ungual treatment, the method comprising:
(a) providing a nail having at least two spaced apart holes formed from a surface thereof and extending into the nail;
(b) topically applying a radiolabeled pharmaceutical composition to the nail, at least a portion of which is received in the at least two holes;
(c) sectioning a portion of the nail proximate the at least two holes into a plurality of sections;
(d) determining, for each of the plurality of sections, a concentration of radioactivity in the section as a function of position with respect to the at least two holes; and
(e) optimizing a spacing between the at least two holes based at least partially on the determinations in step (d).

2. A method of treating an ungual infection in a patient using a topically applied first pharmaceutical composition, the method comprising:
(a) predetermining an optimum spacing between at least two holes according to the method of claim 1 using a second pharmaceutical composition, the second pharmaceutical composition being a radiolabeled version of the first pharmaceutical composition;
(b) forming at least two holes spaced apart by the predetermined optimum spacing in a nail of the patient; and
(c) topically applying the first pharmaceutical composition to the nail of the patient.

3. A method of treating an ungual infection in a patient comprising a step of topically applying a pharmaceutical composition comprising an antifungal agent on an infected nail provided with at least two holes spaced apart.

4. The method according to claim 3, wherein the antifungal agent is terbinafine or a pharmaceutical salt or ester thereof.

5. The method according to claim 3, wherein the at least two holes apart have been provided by applying a method comprising:
(a) providing a nail having at least two spaced apart holes formed from a surface thereof and extending into the nail;
(b) topically applying a radiolabeled pharmaceutical composition to the nail, at least a portion of which is received in the at least two holes;
(c) sectioning a portion of the nail proximate the at least two holes into a plurality of sections;
(d) determining, for each of the plurality of sections, a concentration of radioactivity in the section as a function of position with respect to the at least two holes; and
(e) optimizing a spacing between the at least two holes based at least partially on the determinations in step (d).

6. A pharmaceutical composition comprising an antifungal agent for its use in the treatment of an ungual infection in a patient, comprising the step of topically applying the pharmaceutical composition on an infected nail provided with at least two holes spaced apart.

7. The pharmaceutical composition according to claim 6, wherein the antifungal agent is terbinafine or a pharmaceutical salt or ester thereof.

8. The pharmaceutical according to claim 6, wherein the at least two holes spaced apart have been provided by applying a method comprising:
(a) providing a nail having at least two spaced apart holes formed from a surface thereof and extending into the nail;
(b) topically applying a radiolabeled pharmaceutical composition to the nail, at least a portion of which is received in the at least two holes;
(c) sectioning a portion of the nail proximate the at least two holes into a plurality of sections;
(d) determining, for each of the plurality of sections, a concentration of radioactivity in the section as a function of position with respect to the at least two holes; and
(e) optimizing a spacing between the at least two holes based at least partially on the determinations in step (d).
